(19) United States
(12) Patent Application Publication
(10) Pub. No.: US 2020/0093646 A1
(43) Pub. Date: Mar. 26, 2020

(54) SUPER-ABSORBENT, LOW TRAUMA, ADVANCED WOUND DRESSING

(71) Applicants: KCI Licensing, Inc., San Antonio, TX (US); Systagenix Wound Management, Limited, West Sussex (GB)

(72) Inventors: Christopher B. Locke, Bournemouth (GB); Clinton Hill, Colne (GB)

(21) Appl. No.: 16/527,985

(22) Filed: Jul. 31, 2019

Related U.S. Application Data
(60) Provisional application No. 62/733,846, filed on Sep. 20, 2018.

(51) Int. Cl. A61F 13/02; A61F 13/00; A61B 46/20

(52) U.S. Cl. CPC A61F 13/0209; A61F 13/00029; A61F 13/022; A61L 15/58; A61F 13/0223; A61B 2046/205; A61B 46/20

(57) ABSTRACT

A wound dressing includes a perforated film layer, a hydrophilic foam layer, a drape layer, and superabsorbent projections. The perforated film layer is configured to engage a wound bed and has a first side and a second side, the second side configured to face the wound bed. The hydrophilic foam layer also has a first side and a second side, the second side configured to face the first side of the perforated film layer. The drape layer also has a first side and a second side, the second side configured to face the first side of the hydrophilic foam layer. The hydrophilic foam layer also has a plurality of superabsorbent projections fixed to and extending from the first side of the hydrophilic foam layer towards the second side of the drape layer. The drape layer further has a first drape and a second drape, the first drape comprising an adhesive-coated ring configured to peripherally surround and overlap the second drape.

### CROSS-REFERENCE TO RELATED APPLICATIONS

This application claims the benefit of priority to U.S. Provisional Application No. 62/733,846, filed on Sep. 20, 2018, which is incorporated herein by reference in its entirety.

### BACKGROUND

The present disclosure relates generally to a wound dressing. The present disclosure relates more particularly to a wound dressing having a perforated film layer, a hydrophilic foam layer, and a drape layer, wherein the perforated film layer is placed on the wound bed and the hydrophilic layer includes a plurality of superabsorbent projections extending towards the drape layer.

It is often desirable to remove fluid from a wound bed to promote the healing of the wound. In some cases, wound dressings include a layer of foam configured to absorb the fluid from the wound bed. However, the absorption capacity of the wound dressing is often times quite low, such that not all of the wound fluid is absorbed into the wound dressing.

There is often no consistent way to indicate when the wound dressing has reached its fluid absorption capacity, so often times a caregiver may not replace the wound dressing as often as desired, or the caregiver may replace the wound dressing too frequently which may retard the healing process, cause additional damage to the wound bed, or cause pain to the patient. Additionally, the dressings often release the absorbed fluid upon removal of the dressing back into the wound bed. The foam layer of the wound dressing may be inadequate in retaining the absorbed wound fluid.

Additionally, it is often desirable to have a flexible and conformable wound dressing, such that the dressing can withstand the movement of the patient. However, increasing the absorption of a wound dressing by increasing the size or quantity of the foam layers or superabsorbent sheet often results in a wound dressing that is stiff and unconformable to the patient. Therefore, a compromise is often made as to how absorptive a dressing can be made while still retaining some flexibility of the wound dressing. It is therefore desirable to provide a highly flexible and conformable foam dressing that absorbs and retains high quantities of fluids.

### SUMMARY

One implementation of the present disclosure is a wound dressing including a perforated film layer, a hydrophilic foam layer, a drape layer, and a plurality of superabsorbent projections. The perforated film layer is configured to engage a wound bed, and has a first side and a second side, the second side configured to face the wound bed. The hydrophilic foam layer has a first side and a second side, wherein the second side is configured to face the first side of the perforated film layer. The drape layer has a first side and a second side, wherein the second side is configured to face the first side of the of the hydrophilic foam layer. The plurality of super absorbent projections (nodules, dots, bumps, lumps, islands, protuberances) are fixed to the first side of the hydrophilic foam layer and extend towards the second side of the drape layer. Finally, the drape layer comprises a first drape and a second drape, the first drape comprising an adhesive-coated ring configured to peripherally surround and overlap the second drape.

Another implementation of the present disclosure is a method of making a wound dressing. The method includes providing a perforated film layer configured to engage a wound bed, and having a first side and a second side, the second side configured to face the wound bed. The method further includes providing a hydrophilic foam configured to lay atop the perforated film layer, and having a first side and a second side, the second side configured to face the first side of the perforated film layer. The method further includes preparing a slurry comprising a superabsorbent material. The method further includes applying the slurry onto the first side of the hydrophilic foam layer in the form of a plurality of non-contiguous deposits. The method further includes providing a fusible fiber layer atop the deposits and the first side of the hydrophilic foam layer. The method further includes providing a drape layer having a high moisture vapor transmission rate atop the fusible fiber, wherein the drape layer comprises a first adhesive drape and a second non-adhesive drape, the first drape defining a window and the second drape covering the window and adhered about its periphery to the first drape.

Another implementation of the present disclosure is a method of making a wound dressing. The method includes providing a perforated film layer having a first side and a second side. The method further includes placing a hydrophilic foam layer, having a first side and a second side, atop the perforated film layer, wherein the second side of the hydrophilic layer contacts the first side of the perforated film layer The method further includes placing a non-adhesive drape layer, having a first side and a second side, atop the hydrophilic foam layer, wherein the second side of the non-adhesive drape layer contacts the first side of the hydrophilic foam layer. The method finally includes placing an adhesive drape layer, having a first side, a second side, and a central opening, atop the non-adhesive drape layer, wherein the second side of the non-adhesive drape layer contacts the first side of the non-adhesive drape layer and the non-adhesive drape layer is exposed via the central opening.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a top view of a wound dressing, according to an exemplary embodiment.

FIG. 2 is a bottom view of the wound dressing of FIG. 1, according to an exemplary embodiment.

FIG. 3 is an exploded view illustrating several layers of the wound dressing of FIGS. 1-2, according to an exemplary embodiment.

FIG. 4 is an exploded view illustrating several layers of the wound dressing of FIGS. 1-2, according to an exemplary embodiment.

FIG. 5 is a cross-sectional view of a wound dressing adhered to a surface, according to an exemplary embodiment.

FIGS. 6A-6I show a method of making the wound dressing of FIGS. 1-2, according to an exemplary embodiment.

FIG. 7 is a column graph plotting test results that indicate the moisture vapor transmission rate (MVTR), absorbency, and total fluid handling capacity (TFHC) of several wound dressings, according to an exemplary embodiment.

### DETAILED DESCRIPTION

### Overview

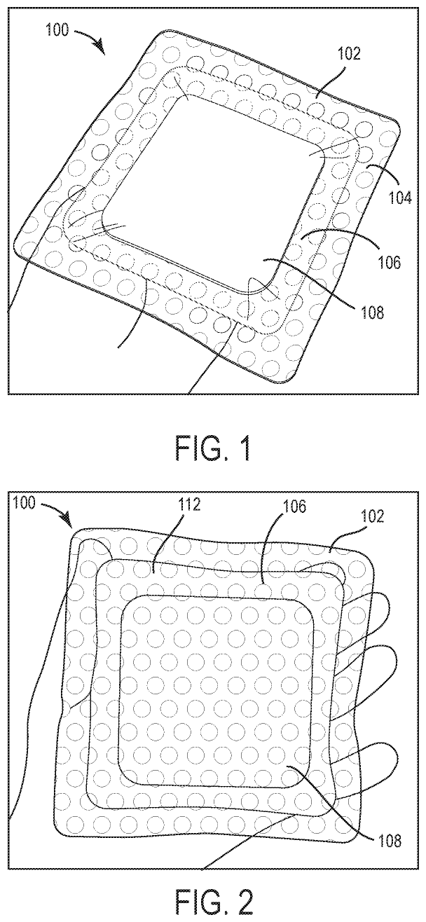

Referring generally to FIGS. 1-7, a wound dressing is shown, according to exemplary embodiments. The wound dressing of the exemplary embodiments has multiple layers including a perforated film layer, a hydrophilic foam layer, and a drape layer. The hydrophilic foam layer includes a plurality of non-contiguous superabsorbent projections (e.g., segments, patches, islands, etc.) configured to draw a wound exudate fluid through the hydrophilic foam layer. In some embodiments, the drape layer is laminated with a fusible fiber to the hydrophilic foam layer.

Advantageously, the superabsorbent projections of the hydrophilic foam layer increase the absorptivity of the wound dressing while retaining flexibility. In some embodiments, the superabsorbent projections comprise a plurality of nodules, dots, bumps, lumps, islands, and protuberances extending from the hydrophilic foam layer towards the drape layer. The superabsorbent projections increase the absorption of the wound dressing to wick wound fluid exudate from the periwound, through the perforated film layer and hydrophilic foam layer, and towards the drape layer. The ordering of the perforated film layer, the hydrophilic foam layer, the super absorbent projections, and the drape layer is configured to establish a relative hydrophilic gradient from the wound bed to the atmosphere through the dressing. Additionally, the drape layer may further act to increase the evaporation rate of wound fluid exudate from the wound dressing. In some embodiments, the drape layer may comprise a first ring-shaped drape layer, adhered over a smaller, second drape layer. In some embodiments, the second drape layer may be free of adhesives, allowing for greater evaporation of wound fluid exudate from the hydrophilic foam layer through to the atmosphere surrounding the drape layer.

Another advantage provided by the wound dressing of the present disclosure is the entrapment of wound fluid exudate within the wound dressing, such that the wound fluid exudate is not released back into the wound bed upon removal of the wound dressing or other movement of the wound dressing. In some embodiments, the drape layer adheres to the perforated film layer around the periphery, such that the hydrophilic foam layer is enclosed within the drape layer and the perforated film layer. This is intended to prevent unintentional release of wound fluid exudate from the hydrophilic foam layer.

Another advantage provided by the wound dressing of the present disclosure is an indication of when the wound dressing has reached its absorption capacity of wound fluid exudate. In some embodiments, the superabsorbent projections are configured to swell upon absorbing the wound exudate fluid, which provides a visual indication of which portion of the wound dressing has absorbed wound exudate and/or when the wound dressing absorbed a maximum capacity of wound exudate. This is intended to prevent unnecessary changing of the wound dressing and provides an indication to a caregiver as to when a wound dressing should be replaced.

Another advantage provided by the wound dressing of the present disclosure is the filtering of undesirable substances by the hydrophilic foam layer. Wound fluid exudate contains a high quantity of salt, which may cause damage and affect the absorptivity of the superabsorbent projections. The hydrophilic foam layer may contain perforations configured to filter and trap the salt from the wound fluid exudate to maintain maximum absorptivity of the superabsorbent projections. Additional features and advantages of the wound dressing are described in detail below.

### Wound Dressing

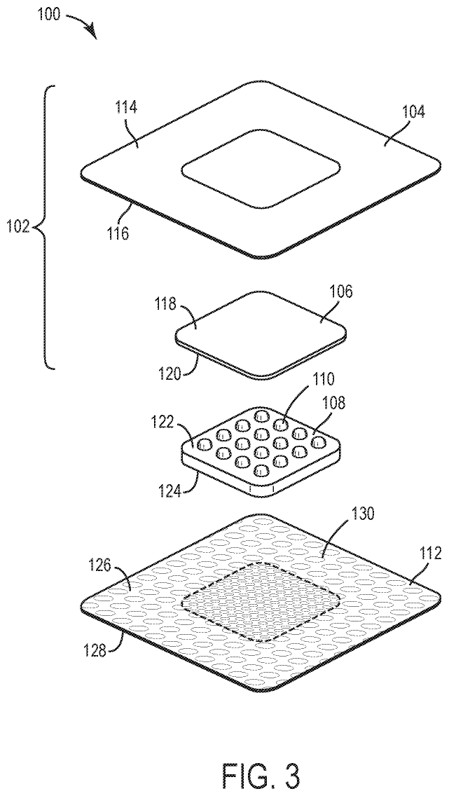

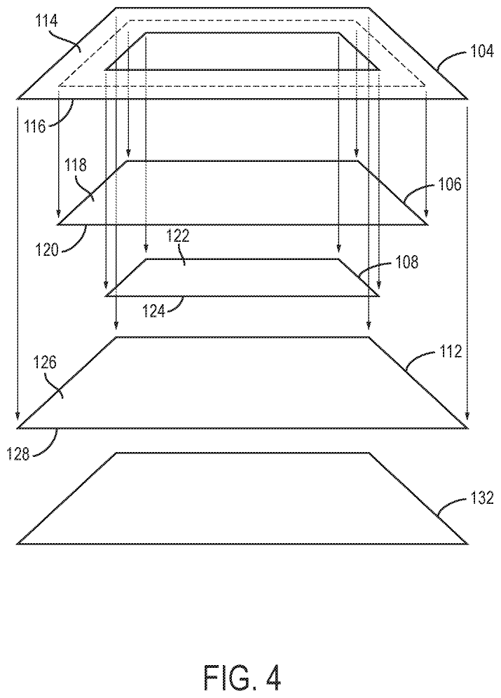

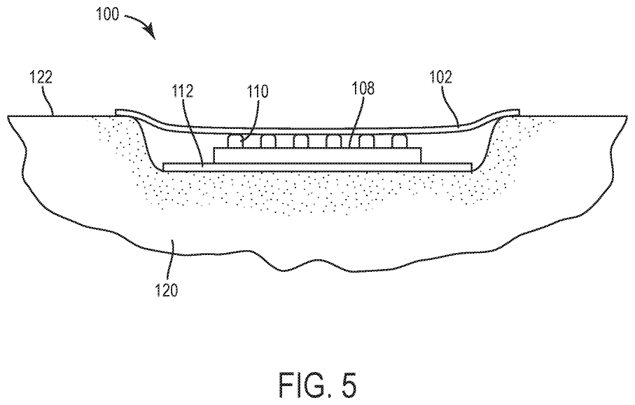

Referring now to FIGS. 1-5 a wound dressing 100 is shown, according to an exemplary embodiment. In a brief overview, FIG. 1 is a top view of the wound dressing 100 as would be visible when wound dressing 100 is adhered to a surface (e.g., a patient's skin). FIG. 2 is a bottom view of wound dressing 100 showing the surface of wound dressing 100 configured to contact a wound. FIG. 3 is an exploded view illustrating several components and layers 102-112 of wound dressing 100. FIG. 4 is an additional exploded view illustrating several components and layers 102-112, 132 of wound dressing 100. FIG. 5 illustrates one embodiment of a cross-sectional view of wound dressing 100 adhered to a patient at a wound bed.

In various embodiments, wound dressing 100 can be formed as a substantially flat sheet for topical applications to wounds, or formed as a contoured dressing for application to body surfaces having high curvature. The size and shape of wound dressing 100 can vary depending on the size of the wound to be dressed and its location. For example, it is contemplated that the size of wound dressing 100 can range from approximately 1 cm² to 100 cm², and more preferably from approximately 4 cm² to 100 cm². However, other shapes and sizes of wound dressing 100 are also possible depending on the intended use. In other embodiments, wound dressing 100 may have a substantially convex or concave shape, or other customizable topography to adhere to wounds located on areas such as the knee or elbow.

Wound dressing 100 is shown to include (among other possible layers) a drape layer 102, a hydrophilic foam layer 108, a plurality of superabsorbent projections 110, and a perforated film layer 112. In the embodiment shown, drape layer 102 includes an adhesive drape layer 104 and a non-adhesive drape layer 106, wherein the adhesive drape layer 104 is formed in a ring-shape and is configured to overlay and overlap the non-adhesive drape layer 106. In some embodiments, drape layer 102 may consist of only a single layer, formed of both adhesive and non-adhesive zones. In some embodiments, the drape layer 102 and the hydrophilic foam layer 108 are bonded to each other (e.g., laminated together using a fusible fiber). In the embodiment shown in FIG. 10, wound dressing 100 is applied for use at the wound bed 136, with the layers 102-112 lying entirely within the confines of the periwound 134. In other embodiments, the drape layer 102 may extend past a perimeter of the perforated film layer 112 and adhere to the top of the periwound 134. However, other configurations of the locations of the layers are also possible depending on the intended use.

### Drape Layer

In some embodiments, wound dressing 100 includes a drape layer 102. Drape layer is shown to include an adhesive drape layer 104 and a non-adhesive drape layer 106. Adhesive drape layer 104 is shown to include a first side 114 and a second side 116 opposite first side 114. Second side 116 is configured to face a wound. When wound dressing 100 is applied to a wound, first side 114 faces away from the wound whereas second side 116 faces towards the wound. In some embodiments, the second side 116 of adhesive drape layer 104 is coated with an adhesive. Second side 116 of adhesive drape layer 116 may be configured to adhere to at least one of non-adhesive drape layer 106 and perforated film layer 112. In the embodiment shown, adhesive drape layer 104 is substantially ring-shaped, or “picture frame” shaped, such that there is a central opening. Thus, when adhesive drape layer 104 is placed over non-adhesive drape layer 106, a central portion of a second side 120 of non-adhesive drape layer 106 remains exposed. In other embodiment, adhesive drape layer 104 may not contain such a central opening, such that adhesive drape layer 104 covers the entirety of non-adhesive drape layer 104. Adhesive drape layer 104 attaches over non-adhesive drape layer 106. In some embodiments, adhesive drape layer 104 overlaps with non-adhesive drape layer 106 within a range of 5 mm to 8 mm peripherally. In some embodiments, the outer perimeter of adhesive drape layer 104 attaches via the adhesive over perforated film layer 112. In some embodiments, second side 116 of adhesive drape layer 106 may contain a release liner to be removed prior to application of the adhesive drape layer 106 over non-adhesive drape layer 104 and perforated film layer 112.

Non-adhesive drape layer 106 is shown to include a first side 118 and a second side 120 opposite first side 118. Second side 120 is configured to face a wound. When wound dressing 100 is applied to a wound, first side 118 faces away from the wound whereas second side 120 faces towards the wound. Non-adhesive drape layer 106 attaches over hydrophilic foam layer 108, such that second side 120 of non-adhesive drape layer 106 contacts a first side 122 of hydrophilic foam layer 108. Second side 120 of non-adhesive drape layer 106 may also contact, in various locations, superabsorbent projections 110. In some embodiments, the imprints made by superabsorbent projections 110 may be seen on first side 118 of non-adhesive drape layer 106, such that first side 118 of non-adhesive drape layer 106 has a plurality of bumps. In some embodiments, non-adhesive drape layer 106 may comprise a release liner on first side 118 to be removed upon application of the non-adhesive drape layer 106, prior to adhering adhesive drape layer 104 over non-adhesive drape layer 106.

In some embodiments, non-adhesive drape layer 106 is substantially free of adhesive. In some embodiments, non-adhesive drape layer 106 may have a thickness substantially within a range of 20 microns to 50 microns. In other embodiments, non-adhesive drape layer 106 may have a thickness less than 20 microns or greater than 50 microns. In the embodiment shown, non-adhesive drape layer 106 lies substantially within the perimeter of adhesive drape layer 104. In other embodiments, non-adhesive drape layer 106 may be of the same size as adhesive drape layer 104. In other embodiments, drape layer 102 may consist of a single layer, such that both the adhesive and non-adhesive portions are contained within a singular drape layer 102.

In some embodiments, drape layer 102 is laminated to hydrophilic foam layer 108 using a fusible fiber positioned between drape layer 102 and hydrophilic foam layer 108. The fusible fiber may act to isolate and maintain the position of the superabsorbent projections 110 between the drape layer 102 and hydrophilic foam layer 108 Drape layer 102 can be bonded to hydrophilic foam layer 108, for example, by an adhesive or by radiation cross-linking. In some embodiments, drape layer 102 is bonded to the hydrophilic foam layer 108 by urethane or urea linkages. This can be achieved by applying drape layer 102 to hydrophilic foam layer 108 (substantially without mixing) before polyurethane curing is complete. In some embodiments, only adhesive drape layer 104 of drape layer 102 may be bonded to hydrophilic foam layer 108. In other embodiments, only non-adhesive drape layer 106 may be bonded to hydrophilic foam layer 108.

In some embodiments, drape layer 102 is a thin layer(s) of polyurethane film. One example of a suitable material for drape layer 102 is the polyurethane film known as ESTANE 5714F. Other suitable polymers for forming drape layer 102 include poly alkoxylalkyl acrylates and methacrylates, such as those described in Great Britain Patent Application No. 1280631A filed Nov. 22, 1002, the entire disclosure of which is incorporated by reference herein. In some embodiments, drape layer 102 includes a continuous layer of a high-density blocked polyurethane foam that is predominantly closed-cell. In some embodiments, adhesive drape layer 104 and non-adhesive drape layer 106 may be of the same material. In other embodiments, adhesive drape layer 104 and non-adhesive drape layer 106 may be of substantially different materials.

Drape layer 102 may be substantially permeable to liquid and moisture vapor. In other words, drape layer 102 may be permeable to both water vapor and liquid water such as wound exudate. Such permeability is intended to facilitate or enhance a hydrophilic gradient from the wound bed 136, through the wound dressing 100, and to the surrounding atmosphere. In some embodiments, drape layer 102 is impermeable to bacteria and other microorganisms. In other embodiments, drape layer 102 is configured to wick moisture from hydrophilic foam layer 108 and superabsorbent projections 110 to first side 114 of adhesive drape layer 104 and first side 118 of non-adhesive drape layer 106, such that it may evaporate into the atmosphere. In some embodiments, drape layer 102 may be substantially hydrophilic and have a high moisture vapor transmission rate, such as to permit evaporation of would exudate/fluid from first side 114 of adhesive drape layer 104 and first side 118 of non-adhesive drape layer 106.

In the embodiment shown, the perimeter of adhesive drape layer 102 is even with the perimeter of perforated film layer 112. In other embodiments, the perimeter of adhesive drape layer 102 may extend beyond (e.g., circumscribe) the perimeter of perforated film layer 112 to provide a margin for wound dressing 100 (e.g., as an “island” dressing) to the skin of a patient adjacent to the wound being treated, and may comprises an adhesive on the second side 116 configured to attach to the wound.

### Hydrophilic Foam Layer

In some embodiments, wound dressing 100 includes hydrophilic foam layer 108. Hydrophilic foam layer is shown to include a first side 122 and a second side 124 opposite the first side 122. First side 122 is configured to face and, in some embodiments, attach to second side 120 of non-adhesive drape layer 106 while second side 124 is configured to face a first side 126 of perforated film layer 112. In some embodiments, first side 122 of hydrophilic foam layer 108 is laminated via fusible fiber to second side 120 of non-adhesive drape layer 106.

Hydrophilic foam layer 108 may comprise a polyurethane foam or a polyethylene foam. In some embodiments, hydrophilic layer 108 includes a flexible plasticized hydrophilic polymer matrix having an internal cellular structure. Several examples of hydrophilic foams which can be used to make hydrophilic foam layer 108 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein. In other embodiments, hydrophilic foam layer 108 may be formed from aromatic or aliphatic precursors. Advantageously, hydrophilic foam layer 108 may provide enhanced absorbency for liquid exudate. This is because the porous structure of the foam provides for rapid uptake of liquid exudate.

Hydrophilic foam layer 108 is shown to include a plurality of superabsorbent projections 110 distributed on the first side 122. In the embodiments shown, superabsorbent projections 110 are substantially evenly distributed along first side 122 of hydrophilic foam layer 108. In other embodiments, superabsorbent projections 110 may be distributed in a non-uniform pattern on first side 122 of hydrophilic foam layer 108. In some embodiments, superabsorbent projections 110 may comprise one or more of nodules, dots, bumps, lumps, islands, protuberances on first side 122 of hydrophilic foam layer 108. Superabsorbent projections 110 may be formed in a range of shapes such as squares, hexagons, hoops, stars, crosses, or a range of lines.

In some embodiments, hydrophilic foam layer 108 may include a plurality of perforations throughout the hydrophilic foam layer 108. Such perforations may act to filter out undesirable substances from wound fluid exudate. For example, wound fluid exudate may contain a high quantity of salt, which is known affect the absorptivity of superabsorbent projections 110. In some embodiments perforations in the hydrophilic foam layer 108 may act to filter out the salt from the wound fluid exudate when the fluid is drawn from the second side 124 to the first side 122 of hydrophilic foam layer 108. In some embodiments, perforations in the hydrophilic foam layer 108 may act to increase the overall flexibility and conformability of wound dressing 100. In some embodiments, the perforations may extend from the first side 122 to the second side 124. In some embodiments, the perforations may have a diameter substantially within a range between 2 mm to 3 mm. In some embodiments, the perforations may be evenly distributed throughout the hydrophilic foam layer 108. In still other embodiments, the perforations may by unevenly distributed through the hydrophilic foam layer 108.

### Superabsorbent Projections

In some embodiments, wound dressing 100 includes a plurality of superabsorbent projections 110 extending from the first side 122 of hydrophilic foam layer 108. A top portion of superabsorbent projections 110 contacts second side 120 of non-adhesive drape layer 120. In some embodiments, superabsorbent projections 110 comprise a plurality of nodules, dots, bums, lumps, islands, or protuberances.

In some embodiments, superabsorbent projections 110 may be formed from or otherwise include a superabsorbent polymer in the form of granules. The superabsorbent polymer may include Luquasorb 1160 or 1161, such as may be commercially available from BASF. The granules may be contained in a water soluble carrier polymer. One example of the water soluble carrier polymer is polyvinylpyrrolidone(PVP). The superabsorbent polymer of the superabsorbent projections 110 and the water soluble polymer may be formed into a slurry or a suspension using an organic solvent. The organic solvent may include propanone or propanol, and may aid in delivery of the superabsorbent projections 110 to the first side 122 of hydrophilic foam layer 108. In some embodiments, to increase the softness of the superabsorbent granules, a plasticizer may be added to the slurry. In one embodiment, the plasticizer may be water. In some embodiments, the slurry to form the superabsorbent projections 110 may have a formulation of 20 parts by mass of PVP, 10 parts by mass of a superabsorbent polymer, 1 part by mass of glycerol, and 100 parts by mass of propanone. In some embodiments, to plasticize the granules, 1 part to 2 parts by mass of water may be added to the slurry mixture. In other embodiments, a water soluble polymer superabsorbent precursor, such as acrylic acid or 2-acrylamido-2-methyl-propanesulfonic acid (AMPS), with suitable UV curing additives, may replace the superabsorbent polymer. Such a precursor may be a relatively low viscosity solution and can be printed onto hydrophilic foam layer 108 and exposed to UV light to form a soft gel, eliminating the need for a plasticizer. In some embodiments, the water soluble polymer superabsorbent precursor may be similar to that used for preparing hydrogel coatings.

The slurry mixture is applied to first side 122 of hydrophilic foam layer 108 to form superabsorbent projections 110. In some embodiments, the slurry may be applied to hydrophilic foam layer 108 through standard printing methods, such as silk screen printing, gravure printing, or by x-y plotter printing. Superabsorbent projections 110 may be in any non-contiguous shapes such as circles, squares, hexagons, hoops/halos, stars, crosses, a range of lines, or any combination of shapes. Superabsorbent projections 110 may be printed such that they are substantially evenly distributed on first side 122. In other embodiments, superabsorbent projections 110 may be printed in an uneven (e.g. non-uniform, random, etc.) pattern on first side 122. In either embodiment, superabsorbent projections 110 are arranged in a non-contiguous manner (i.e. isolated, separated, spaced-apart, non-touching, etc.) so that a region remains between superabsorbent projections 110 to preserve flexibility of wound dressing 100. In some embodiments, superabsorbent projections 110 may include a flexible plasticized hydrophilic polymer matrix having a substantially continuous internal structure. In some embodiments, After the slurry mixture has dried in the pattern of superabsorbent projections 110, the superabsorbent projections 110 may be printed with an adhesive coated film to allow superabsorbent projections 110 to adhere to second side 120 of non-adhesive drape layer 106. In some embodiments, the adhesive applied to superabsorbent projections 110 is moisture vapor transmitting and/or patterned to allow passage of water vapor therethrough. The adhesive may include a continuous moisture vapor transmitting, pressure-sensitive adhesive layer of the type conventionally used for island-type wound dressings (e.g., a polyurethane or polyethylene-based pressure sensitive adhesive). One example of an adhesive which can be used is a pressure sensitive adhesive based on acrylate ester copolymers, polyvinyl ethyl ether and polyurethane, as described in Great Britain Patent Application No. 1280631A. The basis weight of the adhesive may be 20 to 250 g/m², and more preferably 50 to 150 g/m². In some embodiments, after the adhesive has been applied to superabsorbent projections 110, a surface head (i.e., an iron) is applied to non-adhesive drape layer 106 such that non-adhesive drape layer 106 bonds to the superabsorbent projections 110.

Superabsorbent projections 110 may be configured to swell upon absorption of fluid, such as wound fluid exudate. Superabsorbent projections 110 may be more hydrophilic than hydrophilic foam layer 108 and perforated film layer 112, such that superabsorbent projections 110 assist in wicking fluid from the second side of the hydrophilic foam layer 124 towards the first side 122 and into the superabsorbent projections 110. Such high hydrophilicity of superabsorbent projections 110 establishes a hydrophilic gradient from the wound bed 136 to the atmosphere surrounding wound dressing 100. Each of the superabsorbent projections 110 may absorb and swell upon absorption of fluid, providing a visual indication of which portion of wound bed 136 has absorbed fluid. In some embodiments, even upon absorption of fluid each of the superabsorbent projections 110 will remain isolated.

### Perforated Film Layer

In some embodiments, wound dressing 100 includes a perforated film layer 112. Perforated film layer 112 is shown to include a first side 126 and a second side 128 opposite first side 126. Second side 128 is shown to contact wound bed 136. In some embodiments, first side 126 of perforated film layer 112 comprises a release liner, which may be removed upon application of hydrophilic foam layer 108 to the perforated film layer 112. In the embodiment shown in FIG. 10, perforated film layer 112 and drape layer 102 lie entirely within the confines of the wound bed 136. In other embodiments, drape layer 102 may extend past perforated film layer 112 and may adhere to a top surface of the periwound 134. When wound dressing 100 is applied to a wound, first side 126 faces away from the wound whereas second side 128 faces towards the wound. In some embodiments, the periphery of first side 126 is adhered to second side 116 of adhesive drape layer 104, forming a seal around the remaining layers of wound dressing 100 (non-adhesive drape layer 106, hydrophilic foam layer 108, and superabsorbent projections 110).

Perforated film layer 112 may comprise a polyurethane foam or a polyethylene foam. In some embodiments, perforated film layer 112 may comprise a trilaminate layer. In some embodiments first side 126 of perforated film layer 112 may comprise an acrylic coating or adhesive. First side 126 with an acrylic coating/adhesive may attach to second side 116 of adhesive drape layer 104 and second side 124 of hydrophilic foam layer 108. In some embodiments, first side 126 may comprise an even layer of acrylic coating/adhesive on the entirety of first side 126. In other embodiments, first side 126 may have a patterned layer of acrylic coating/adhesive, or may be such that the acrylic coating/adhesive covers only a portion of first side 126. In some embodiments, second side 128 of perforated film layer 112 may comprise a silicone coating, such as a silicone gel. Inclusion of a silicone coating on second side 128 of perforated film layer 112 may allow for the removal and repositioning of the wound dressing 100 after application of wound dressing 100. In some embodiments, the silicone coating may extend on second side 128 of perforated film layer 112 only in a central region, such that it is underneath the portion of perforated film layer 112 attached to hydrophilic foam layer 108. A periphery of second side 128 of perforated film layer 112 may be free of the silicone coating. In other embodiments, the silicone coating may extend on the entirety of second side 128 of perforated film layer 112.

In some embodiments, the silicone coating on second side 128 of perforated film layer 112 may comprise a plurality of perforations. Such perforations may increase flexibility of the wound dressing 100 and increase the uptake of wound fluid exudate into the perforated film layer 112. In some embodiments, perforations in the silicone coating in a central region on second side 128 of perforated film layer 112 may have a diameter substantially within a range between 2 mm to 5 mm. In some embodiments, perforations in the silicone coating in a peripheral region on second side 128 of perforated film layer 112 may have a diameter substantially within a range between 7 mm to 9 mm. In some embodiments, perforations in the silicone on second side 128 of perforated film layer 112 may be evenly distributed. In other embodiments, perforations in the silicone on second side 128 of perforated film layer 112 may be distributed in an uneven pattern.

### Method of Making a Wound Dressing

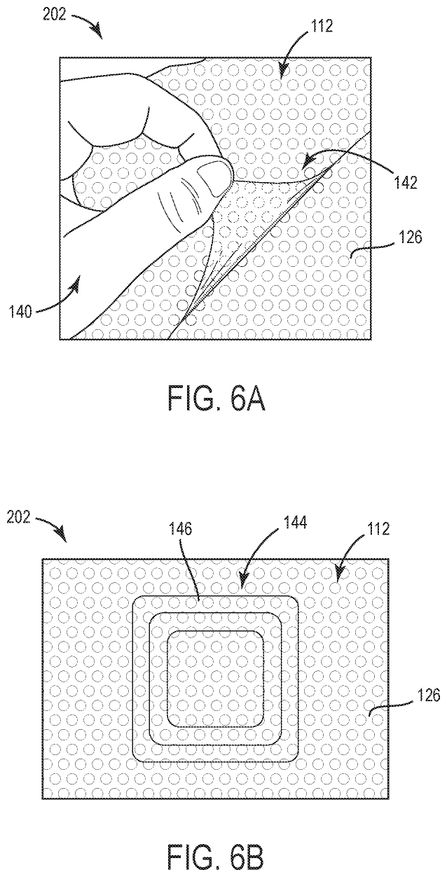

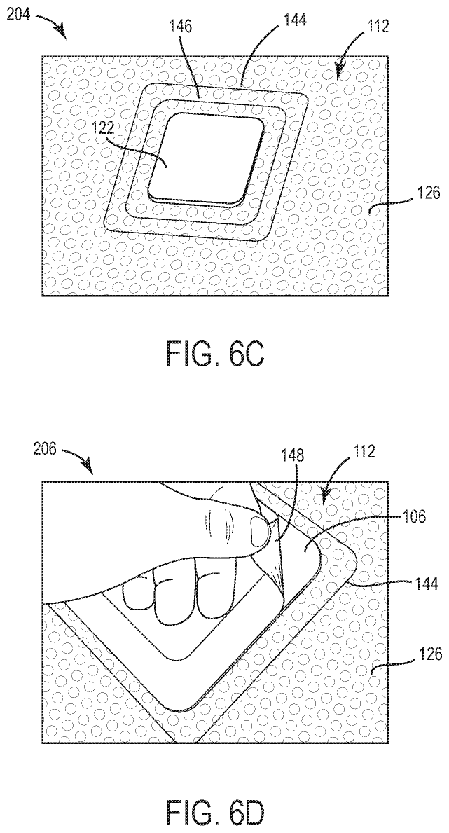

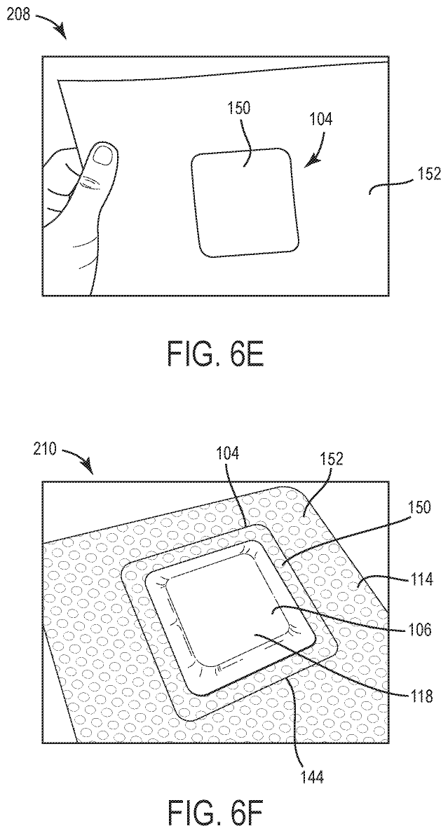

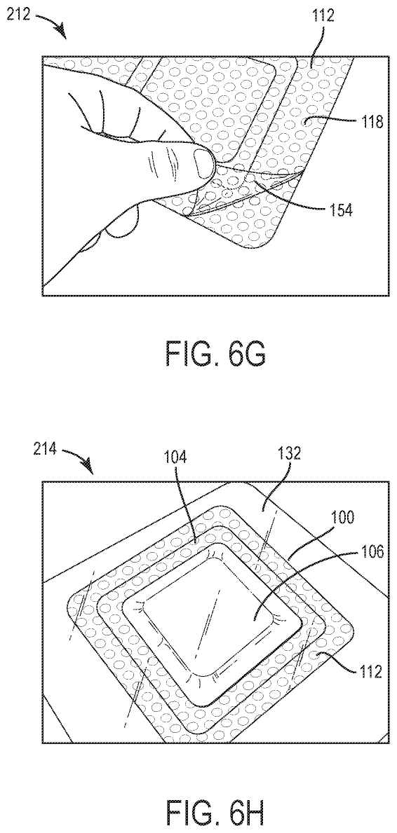

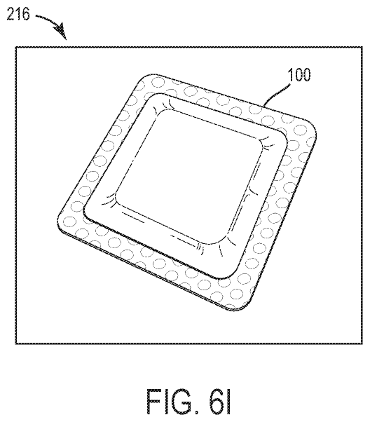

Referring now to FIGS. 6A-6I, a method of making the wound dressing 100 is shown, according to an exemplary embodiment. The first step 200 in making the wound dressing 100 begins with the perforated film layer 112. In some embodiments, the first side 126 of perforated film layer 112 is covered by a release liner 142, which acts to protect the perforated film layer 112 prior to use. As seen in FIG. 6A, the first step 200 is to place the perforated film layer 112 with the second side 128 facing downwards and remove, by a user 140, the release liner 142 from the first side 126 of the perforated film layer. In some embodiments, removal of the release liner 142 exposes the acrylic coating on the first side 126 of the perforated film layer 112. In step 202, as seen in FIG. 6B, the perforated film layer 112 is placed over a template 144, such that the template 144 is in contact with and below the second side 128 of perforated film layer 112. The template 144 includes a plurality of outlines 146 to assist in the placement of various layers of the wound dressing 100.

As seen in FIG. 6C, in step 204 the hydrophilic foam layer 108 is placed, with the second side 124 facing down, above the perforated film layer 112, such that the second side 124 of the hydrophilic foam layer 108 contacts the first side 126 of the perforated film layer 112. In the embodiment shown, the first side 122 of the hydrophilic foam layer 108 is upward-facing and exposed. Additionally, the hydrophilic foam layer 108 is placed within the template 144, such that the hydrophilic foam layer 108 lies within a central outline 146 of the template. In step 206, as seen in FIG. 6D, the non-adhesive drape layer is placed 106 above the hydrophilic foam layer 108. In the embodiment shown, the second side 120 of non-adhesive drape layer 106 is placed above and contacts the first side 122 of hydrophilic foam layer 108. In some embodiments, the first side 122 of hydrophilic foam layer 108 may comprise a plurality of superabsorbent projections 110, such that the superabsorbent projections 110 contact the second side 120 of non-adhesive drape layer 106. In the embodiment shown, the non-adhesive drape layer 106 extends beyond a perimeter of the hydrophilic foam layer 108, such that the outline 146 of the template 144 used for the non-adhesive drape layer 106 is around the perimeter of the hydrophilic foam layer 108. As shown in FIG. 6D, a release liner 148 of the non-adhesive drape layer 106 is also removed from the first side 118 of non-adhesive drape layer 106. The release liner 148 may serve to cover and protect the first side 118 of the non-adhesive drape layer 106 prior to release.

In step 208, as seen in FIG. 6E, the adhesive drape layer 104 is prepared. In the embodiment shown, a central hole 150 is cut in a sheet of adhesive material 152 to form the adhesive drape layer 104. The adhesive drape layer 104 has a central hole 150 forming a window in the central portion of the adhesive drape layer 104. In step 210, as seen in FIG. 6F, the adhesive drape layer 104 is placed over the non-adhesive drape layer 106 and other layers of the wound dressing 100. The second side 116 of adhesive drape layer 104 faces downwards and contacts the first side 118 of the non-adhesive drape layer 106. The adhesive drape layer 104 peripherally surrounds the non-adhesive drape layer 106, such that the adhesive drape layer 106 extends through the central hole 150 in the sheet of adhesive material 152. In some embodiments, the adhesive drape layer 104 may partially or fully overlap with the non-adhesive drape layer 106. In various embodiments, the adhesive drape layer 104 and/or the non-adhesive drape layer 106 may be laminated via a fusible fiber to the hydrophilic foam layer.

In step 212, as seen in FIG. 6G, the wound dressing 100 is removed from the template 144 and flipped over to expose the second side 118 of the perforated film layer 112. The user 140 removes the release liner 154 from the second side 118 of the perforated film layer 112. In some embodiments, removal of the release liner 154 exposes the silicone coating on the second side 118 of the perforated film layer 112. In step 214, as seen in FIG. 6H, the exposed silicone coating on the second side 118 of the perforated film layer 112 is placed, downwards-facing, onto an oriented polypropylene (OPP) liner 132. In other embodiments, other materials may be used for liner 132. In step 216, the wound dressing 100 is cut out from the OPP liner 132 by the user 140, to form the final wound dressing 100 as seen in FIG. 6I. In some embodiments, the template 144 may be used to aide in cutting the OPP liner 132 to ensure wound dressing 100 is of the proper size.

### Test Results

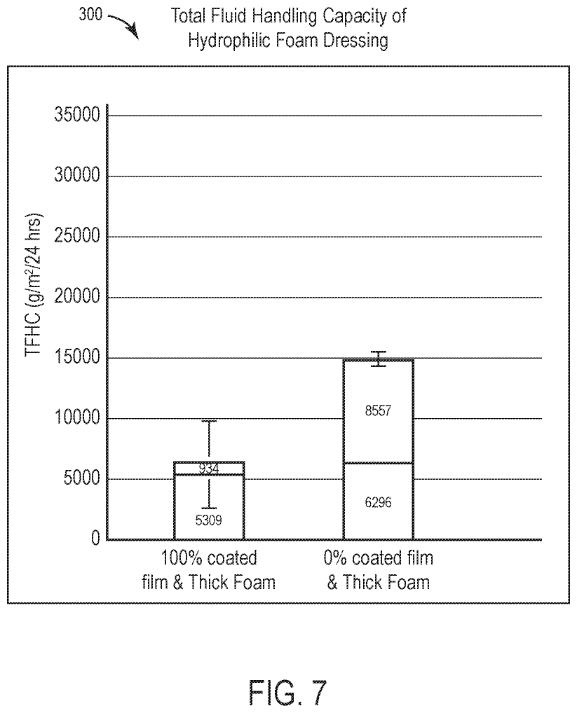

Referring now to FIG. 7, a graph illustrating the performance of wound dressing 100 relative to similar wound dressings wherein the hydrophilic foam layer 108 is placed beneath a 100% adhesive-coated drape layer is shown, according to an exemplary embodiment. FIG. 18 is a column graph 300 plotting the test results that indicate the moisture vapor transmission rate (MVTR), absorbency, and total fluid handling capacity (TFHC) of two samples of wound dressings. MVTR, absorbency, and TFHC are measured in units of

$$\frac{g}{{m}^{2} \cdot 24 hours} .$$

The TFHC is defined as the sum of MVTR and absorbency (i.e., MVTR+absorbency=TFHC). In the first wound dressing, the hydrophilic foam layer 108 was covered by an acrylic adhesive coated drape layer. In the second wound dressing, the hydrophilic foam layer 108 was covered by a non-adhesive drape layer. As shown in FIG. 18, the wound dressing with the hydrophilic foam layer 108 with the adhesive-coated drape layer has a MVTR of

$$5 , 309 \frac{g}{{m}^{2} \cdot 24 hours} ,$$

an absorbency of

$$934 \frac{g}{{m}^{2} \cdot 24 hours} ,$$

### and a TFHC of

$$6 , 243 \frac{g}{{m}^{2} \cdot 24 hours} .$$

The wound dressing with the hydrophilic foam layer with a non-adhesive drape layer has a MVTR of

$$6 , 296 \frac{g}{{m}^{2} \cdot 24 hours} ,$$

an absorbency of

$$8 , 557 \frac{g}{{m}^{2} \cdot 24 hours} ,$$

### and a TFHC of

$$14 , 853 \frac{g}{{m}^{2} \cdot 24 hours} .$$

The results show that the inclusion of an acrylic adhesive coated drape layer in the wound dressing as compared to a non-adhesive drape layer significantly reduces the MVTR and the absorbency. Thus, the inclusion of non-adhesive drape layer 106 within wound dressing 100 with the adhesive drape layer 104 surrounding only a perimeter of, the wound dressing 100 is believed to increase the MVTR and absorbency as compared to a standard wound dressing covered entirely by an adhesive drape layer. It is also expected that the inclusion of superabsorbent projections 110 in wound dressing 100 would act to further increase the MVTR and absorbency as compared to the test results, in with there was no super-absorbent layer between the foam and the drape layer.

According to an exemplary embodiment, wound dressing 100 has multiple advantages over previous wound dressings. Wound dressing 100 is both soft and flexible, and is capable of wicking fluid away from wound bed 136 and absorbing fluid over the plurality of non-contiguous superabsorbent projections 110. As the superabsorbent projections 110 are located above hydrophilic foam layer 108 and perforated film layer 112, fluid is absorbed at a separate location so as to minimize a level of fluid that remains close to wound bed 136. Further, the non-contiguous superabsorbent projections 110 may be isolated from the edges of wound dressing 100 to reduce the risk of fluid leaking from wound dressing 100. The drape layer 102 and perforated film layer 112 adhere to form a seal surrounding the hydrophilic foam layer 108 and superabsorbent projections 110 to further reduce the risk of fluid leaking from wound dressing 100. Further, the inclusion of a silicone coating within the perforated film layer 112 aids in removal and repositioning of the wound dressing 100 after wound dressing 100 has been applied.

According to an exemplary embodiment, the superabsorbent polymer in the form of granules has a higher absorption capacity than traditionally used absorbent non-woven dressings and therefore can maintain the same absorption capacity as a traditional dressing with less material required. With less granules and material needed for the superabsorbent projections 110, the overall structure of wound dressing 110 is highly flexible and a thickness of wound dressing 100 may be reduced. Further, the concentration of superabsorbent polymer within the slurry may be increased to increase the absorption capacity of wound dressing 100 to have a high storage and fluid management capacity, or the print pattern and density of superabsorbent projections 110 may be changed to easily adapt the wound dressing 100 for a particular wound. Additionally, the swelling of superabsorbent projections 110 provides a visual indicator as to when wound dressing 100 has reached maximum fluid capacity and when wound dressing 100 needs to be changed.

According to an exemplary embodiment, the drape layer 102 allows for a greater evaporation rate of fluid while maintaining both a fluid seal and overall flexibility of wound dressing 100. Adhesive drape layer 106 forms a sealing ring when attached to perforated film layer 112, such that fluid is ‘locked’ within the seal to prevent leaks. However, adhesive drape layer 106, has a central opening, such that non-adhesive drape layer 104 is exposed to the atmosphere. As non-adhesive drape layer 104 is substantially free of adhesives, the evaporation of fluid from within wound dressing 100 is greater over the surface of non-adhesive drape layer 104 than adhesive drape layer 106. The reduction in size of adhesive drape layer 106 to just the periphery of wound dressing 100 allows for overall greater evaporation of fluid from within wound dressing 100, providing a greater overall absorption of wound dressing 100.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A wound dressing comprising:
a perforated film layer configured to engage a wound bed, and having a first side and a second side, wherein the second side is configured to face the wound bed;
a hydrophilic foam layer having a first side and a second side, the second side configured to face the first side of the perforated film layer;
a drape layer having a first side and a second side, the second side configured to face the first side of the of the hydrophilic foam layer; and
a plurality of superabsorbent projections fixed to and extending from the first side of the hydrophilic foam layer towards the second side of the drape layer,
wherein the drape layer comprises a first drape and a second drape, the first drape comprising an adhesive-coated ring configured to peripherally surround and overlap the second drape.

2. The wound dressing of claim 1, wherein the second drape is substantially free of adhesives.

3. The wound dressing of claim 2, wherein the first drape forms an adhesive border substantially surrounding the non-adhesive second drape, and the non-adhesive second drape substantially overlies the hydrophilic foam layer and the superabsorbent projections.

4. The wound dressing of claim 1, wherein the first drape overlaps the second drape substantially within a range between 5 mm to 8 mm.

5. The wound dressing of claim 1, wherein the drape layer is laminated with fusible fiber to the hydrophilic foam layer.

6. The wound dressing of claim 1, wherein the drape layer is laminated with fusible fiber to the hydrophilic foam layer and the superabsorbent projections.

7. The wound dressing of claim 1, wherein the superabsorbent projections are operable to draw a wound exudate fluid through the hydrophilic foam layer.

8. The wound dressing of claim 1, wherein the drape layer comprises a material having a high moisture vapor transmission rate, and is configured to permit evaporation of the wound exudate fluid from the superabsorbent projections.

9. The wound dressing of claim 8, wherein the ordering of the perforated film layer, the hydrophilic foam layer, the superabsorbent projections, and the drape layer, are configured to establish a relative hydrophilic gradient from the wound bed to an ambient atmosphere through the dressing.

10. The wound dressing of claim 1, wherein the superabsorbent projections are printed onto the first side of the hydrophilic elastic foam layer by screen printing, gravure printing, or by x-y plotter printing.

11. The wound dressing of claim 1, wherein the superabsorbent projections comprise a flexible plasticized hydrophilic polymer matrix having a substantially continuous internal structure.

12. The wound dressing of claim 11, wherein the superabsorbent projections comprise a polymer in the form of granules contained in a water soluble carrier polymer such as polyvinylpyrrolidone.

13. The wound dressing of claim 1, wherein the hydrophilic foam layer comprises a plurality of perforations.

14. The wound dressing of claim 1, wherein the first side of the perforated film layer comprises an acrylic adhesive configured to adhere the first side of the perforated film layer to the second side of the drape layer, and the second side of the perforated film layer comprises a silicone coating.

15. The wound dressing of claim 14, wherein the silicone coating comprises a plurality of perforations, such that perforations in a central region of the silicone coating have a diameter substantially within a range between 2 mm and 5 mm, and perforations in a peripheral region of the silicone coating have a diameter substantially within a range between 7 mm and 9 mm.

16. A method of making a wound dressing comprising:
providing a perforated film layer configured to engage a wound bed, and having a first side and a second side, the second side configured to face the wound bed;
providing a hydrophilic foam configured to lay atop the perforated film layer, and having a first side and a second side, the second side configured to face the first side of the perforated film layer;
preparing a slurry comprising a superabsorbent material;
applying the slurry onto the first side of the hydrophilic foam layer in the form of a plurality of non-contiguous deposits;
providing a fusible fiber layer atop the deposits and the first side of the hydrophilic foam layer; and
providing a drape layer having a high moisture vapor transmission rate atop the fusible fiber, wherein the drape layer comprises a first adhesive drape and a second non-adhesive drape, the first drape defining a window and the second drape covering the window and adhered about its periphery to the first drape.

17. The method of claim 16, further comprising the step of arranging the drape layer with the second drape and the window substantially covering the non-contiguous deposits of the superabsorbent material.

18. The method of claim 16, wherein the deposits are configured to swell upon absorbing the wound exudate fluid to provide a visual indication of which portion of the wound dressing is receiving wound exudate and/or when the wound dressing is full.

19. The method of claim 16, wherein the slurry comprises a flexible plasticized polymer matrix hydrophilic polymer matrix having a substantially continuous internal structure.

20. A method of making a wound dressing comprising:
providing a perforated film layer having a first side and a second side;
placing a hydrophilic foam layer, having a first side and a second side, atop the trilaminate perforated film layer, wherein the second side of the hydrophilic layer contacts the first side of the trilaminate perforated film layer;
placing a non-adhesive drape layer, having a first side and a second side, atop the hydrophilic foam layer, wherein the second side of the non-adhesive drape layer contacts the first side of the hydrophilic foam layer; and
placing an adhesive drape layer, having a first side, a second side, and a central opening, atop the non-adhesive drape layer, wherein the second side of the non-adhesive drape layer contacts the first side of the non-adhesive drape layer and the non-adhesive drape layer is exposed via the central opening.

21. The method of claim 20, wherein the adhesive drape layer forms an adhesive border substantially surrounding the non-adhesive drape layer, and the non-adhesive drape layer substantially overlies the hydrophilic foam layer.

22. The method of claim 20, wherein the first side of the hydrophilic foam layer comprises a plurality of superabsorbent projections fixed to and extending from the first side towards the second side of the non-adhesive drape layer.

23. The method of claim 20, wherein the ordering of the perforated film layer, the hydrophilic foam layer, the non-adhesive drape layer, and the adhesive drape layer, are configured to establish a relative hydrophilic gradient from the wound bed to an ambient atmosphere through the dressing.

24. The method of claim 20, further comprising placing the adhesive drape layer over the non-adhesive drape layer such that the adhesive drape layer overlaps the non-adhesive drape layer substantially within a range between 5 mm to 8 mm.

25. The method of claim 20, further comprising laminating, via a fusible fiber, at least one of the adhesive drape layer and the non-adhesive drape layer to the hydrophilic foam layer.
